(19) United States
(12) Patent Application Publication
(10) Pub. No.: US 2010/0028419 A1
(43) Pub. Date: Feb. 4, 2010

(54) MEMBRANE(S) AND USES THEREOF

(76) Inventors: Yuru Deng, Singapore (SG); Zakaria Almsherqi, Singapore (SG); Markus R. Wenk, Singapore (SG); Sepp Dieter Kohlwein, Graz (AT)

Correspondence Address: SEED INTELLECTUAL PROPERTY LAW GROUP PLLC, 701 FIFTH AVE, SUITE 5400, SEATTLE, WA, 98104, US

(21) Appl. No.: 12/527,386

(22) PCT Filed: Feb. 15, 2008
(86) PCT No.: PCT/SG08/00055

§ 371 Date: Aug. 14, 2009

Related U.S. Application Data
(60) Provisional application No. 60/890,081, filed on Feb. 15, 2007.

(51) Int. Cl. A61K 9/127

(52) U.S. Cl. 424/450

(57) ABSTRACT

There is provided at least one agent delivery vehicle comprising at least one membrane of cubic structure or derivable from a membrane of cubic structure, methods for preparing them, and their use for transport of at least one foreign agent. In particular, the agent delivery vehicle may be in the form of at least one organelle, agent delivery particle and/or liposome. The invention further provides kits and methods of treatment using the agent delivery vehicle(s).

### FIELD OF THE INVENTION

The present invention generally relates to agent delivery vehicles comprising at least one membrane, methods for preparing them, and their use for transport of at least one foreign agent. In particular, the agent delivery vehicle may be in the form of at least one organelle, agent delivery particle and/or liposome.

### BACKGROUND OF THE ART

A variety of methods have been developed to facilitate transfer of foreign agents into specific cells. One of the more complicated foreign agents for delivery would be nucleic acids (e.g. genes) due to their inherent negative charge and large size. There are however several methods which are useful for both in vivo and in vitro gene transfer. In the former, a gene is directly introduced (intravenously, intraperitoneally, aerosol, etc.) into a subject. In in vitro gene transfer, the gene is introduced into cells after removal of the cells from specific tissue of an individual. The transfected cells are then introduced back into the subject.

Delivery systems for achieving in vivo and in vitro gene therapy include viral vectors, such as retroviral vectors or adenovirus vectors, microinjection, electroporation, protoplast fusion, calcium phosphate, and liposomes. However, the use of viral vectors creates out-of-control immune responses resulting in deadly outcomes for patients who use them. Liposome vesicles which are known to be taken up by cells via endocytosis result in the liposomes entering the lysosomal degradation pathway. Thus, many liposomes and the foreign agents they carry end up being degraded before reaching the target in the cells.

Cubosomes® are nanoparticles with liquid crystalline phases made from bicontinuous cubic phase dispersions that may be used for delivery of drugs in the form of peptides, proteins, nicotine and the like. However, the potential of Cubosomes® being used as a gene delivery system for delivery of DNA is likely to be restricted and inefficient. In addition to forming pores that are too small for the delivery of DNA which is generally much larger in size, the ability of Cubosomes® to interact with DNA is largely dependent on the charge interaction between negatively charged DNA molecules and cationic surfactants forming the cubic phases. In this respect, Cubosomes® and liposomes share the same mechanism of interaction with DNA molecules and thus have the same problems.

Furthermore, it has been shown that systemic administration of cationic liposome/nucleic acid complexes leads to their facile entrapment in the lung without reaching their target organ. Also, cationic liposomes are cleared too rapidly, and present a host of safety concerns which include the inducement of rampant cell death mainly due to the cytotoxicity of the cationic liposomes. There is thus a need for a safe, efficient and versatile means for transfer of foreign agents into target cells.

Membranes of cubic structure are 3-dimensional nano-periodic structures that naturally occur in a wide variety of living systems. They are based on highly curved surfaces that are mathematically analogous to triply periodic minimal surfaces used in describing both crystalline and liquid crystalline materials at a variety of length scales. These membranes though closely related to the variety of mesoscopic phases (e.g. hexagonal phase or various cubic phases) that result from mixing, dispersion and homogenization of lipids in water outside of the cell are naturally occurring. Membranes of cubic structure have been observed in numerous cell types of all kingdoms of life and in virtually any membrane-bound cell organelle, especially smooth endoplasmic reticulum, plasma membrane, inner nuclear membrane, mitochondrial inner membrane and chloroplast thylakoid membrane. However, knowledge about formation and function of non-lamellar, cubic structures in biological systems is scarce and research so far is restricted to the descriptive level.

For the forgoing reasons, there still remains a significant need to develop a suitable delivery vehicle to provide a safe, versatile and efficient packaging and releasing means for the delivery of any foreign agent into a cell having overcome the problems mentioned above. Accordingly, there is a need to further understand the functional roles of structures and particles for the rational design of lipid-based intracellular drug, proteins and/or gene delivery systems.

### SUMMARY OF THE INVENTION

The present inventors have surprisingly found that membranes of cubic structure may be used in preparing agent vehicle particles, systems or devices.

In particular, the present invention satisfies the need for a suitable delivery vehicle using a membrane of cubic structure to provide a safe, versatile and efficient packaging and releasing means for the delivery of any foreign agent into a cell. The present invention also provides a better understanding of the functional roles of the membranes of cubic structure and provides means to exploit the polymorphic phase behaviour of lipids from the membranes of cubic structure for the rational design of lipid-based intracellular drug and gene delivery systems.

According to the first aspect, the present invention provides at least one agent delivery particle comprising at least one biological membrane and at least one foreign agent. The agent delivery particle may be obtained from at least one organelle comprising at least one membrane and at least one foreign agent. In particular, the agent delivery particle may be a result of fragmentation of the organelle. More in particular, the organelle may comprise at least one membrane of cubic structure prior to contact with the foreign agent.

The agent delivery particle according to any aspect of the present invention may be for use in medicine. In particular, the agent delivery particle of the present invention may be for use in gene therapy.

The foreign agent may be selected from the group consisting of hydrophilic drug, hydrophobic drug, nucleic acid, polypeptide, polysaccharide, virus, and vitamin. In particular, the foreign agent may be nucleic acid.

According to another aspect, the present invention provides at least one isolated organelle comprising at least one membrane and at least one foreign agent. In particular, the isolated organelle may comprise at least one membrane of cubic structure prior to contact with the foreign agent. The isolated organelle of the present invention may be selected from the group consisting of chloroplast, Golgi apparatus, mitochondrion, nucleus, rough endoplasmic reticulum (RER), smooth endoplasmic reticulum (SER), vacuole, and vesicle. In particular, the isolated organelle may be mitochondrion.

The isolated organelle of the present invention may be isolated from any suitable source, for example from at least one amoeba. In particular, the amoeba may be Chaos carolinense.

According to another aspect the present invention provides at least one method of preparing at least one organelle comprising at least one membrane of cubic structure by contacting at least one cell comprising the organelle with at least one fatty acid. In particular, the fatty acid may be fatty acid C22:5.

According to a further aspect, the present invention provides at least one method of preparing at least one organelle comprising at least one foreign agent, comprising the steps of:
(a) isolating at least one organelle from at least one cell; and
(b) contacting the organelle with at least one foreign agent.

In particular, the isolated organelle of step (a) may comprise at least one membrane of cubic structure prior to contacting with the foreign agent. In particular, the isolated organelle may be selected from the group consisting of chloroplast, Golgi apparatus, mitochondrion, nucleus, rough endoplasmic reticulum (RER), smooth endoplasmic reticulum (SER), vacuole, and vesicle. More in particular, the isolated organelle may be mitochondrion. The isolated organelle of step (a) may be isolated from any suitable source, for example from at least one amoeba. In particular, the amoeba may be Chaos carolinense.

According to a further aspect, the present invention provides at least one method of preparing at least one agent delivery particle, comprising the steps of:
(a) isolating at least one organelle from at least one cell;
(b) contacting the organelle with at least one foreign agent; and
(c) preparing at least one agent delivery particle from the organelle comprising the foreign agent of step (b), wherein the obtained agent delivery particle comprises at least one biological membrane and the foreign agent.

In particular, the organelle isolated in step (a) may comprise at least one membrane of cubic structure. More in particular, the agent delivery particle of step (c) may be prepared by fragmenting the organelle comprising the foreign agent of step (b).

The isolated organelle of step (a) may be selected from the group consisting of chloroplast, Golgi apparatus, mitochondrion, nucleus, rough endoplasmic reticulum (RER), smooth endoplasmic reticulum (SER), vacuole, and vesicle. In particular, the isolated organelle may be mitochondrion. More in particular, the isolated organelle of step (a) may be isolated from any suitable source, for example from at least one amoeba. In particular, the amoeba may be Chaos carolinense.

According to a further aspect, the present invention provides at least one agent delivery particle obtainable by the method(s) described above.

According to a further aspect, the present invention provides at least one liposome comprising at least one lipid membrane of cubic structure. The liposome of the present invention may be made from at least one lipid of at least one organelle comprising at least one membrane of cubic structure. The lipids of the liposome of the present invention may be from the organelle selected from the group consisting of chloroplast, Golgi apparatus, mitochondrion, nucleus, rough endoplasmic reticulum (RER), smooth endoplasmic reticulum (SER), vacuole, and vesicle.

According to another aspect, the present invention provides at least one liposome, wherein the lipid membrane of the liposome comprises at least one lipid selected from the group consisting of plasmalogen phosphatidylcholine (PC), plasmalogen phosphatidylethanolamine (PE) and -diacyl phosphatidylinositol (PI). The lipid(s) may be isolated from the cubic membrane according to any aspect of the invention or obtained from a commercial supplier.

The liposome of the present invention may comprise at least one foreign agent. The liposome of the present invention may be for use in delivery of the foreign agent into at least one cell in vivo or in vitro. In the liposome of the present invention, the foreign agent may be selected from the group consisting of hydrophilic drug, hydrophobic drug, nucleic acid, polypeptide, polysaccharide, virus, and vitamin. In particular, the foreign agent may be nucleic acid.

The liposome of the present invention may be for use in medicine. In particular, the liposome of the present invention may be for use in gene therapy.

According to a further aspect, the present invention provides at least one method of preparing the liposome of the present invention comprising the steps of:
(a) isolating at least one organelle comprising at least one membrane of cubic structure;
(b) isolating lipids from the membrane; and
(c) preparing at least one liposome with at least one lipid membrane of cubic structure by contacting at least one lipid of step (b) with at least one aqueous medium.

According to another aspect, the present invention provides at least one method of preparing the liposome of the present invention by contacting at least one lipid selected from the group consisting of plasmalogen phosphatidylcholine (PC), plasmalogen phosphatidylethanolamine (PE) and diacyl phosphatidylinositol (PI) with at least one aqueous medium.

According to another aspect, the present invention provides at least one method of delivery of at least one foreign agent comprising either contacting the agent delivery particle or the liposome of the present invention with at least one cell in vivo or in vitro.

According to another aspect, the present invention provides at least one use of the organelle of the present invention for preparing at least one agent delivery particle for treating at least one subject. In particular, the subject may be at least one human in need of therapy for, or susceptible to, at least one condition or sequelae of the condition.

According to another aspect, the present invention provides at least one use of the liposome of the present invention for preparing at least one medicament for treating at least one subject. In particular, the subject may be at least one human in need of therapy for, or susceptible to, at least one condition or sequelae of the condition.

According to still another aspect, the present invention provides at least one kit for delivery of at least one foreign agent, the kit comprising the organelle, the agent delivery particle, and/or the liposome of the present invention.

According to a further aspect, the present invention provides at least one method of detecting and/or monitoring the presence of at least one pathology in at least one cell, comprising detecting an increase of cubic ER membrane compared to a control, the increase correlating to the presence of the pathology.

According to another aspect, the present invention provides at least one method of gene therapy comprising administering at least one subject with the agent delivery particle of the present invention comprising nucleic acid as the foreign agent, or the liposome of the present invention comprising nucleic acid as the foreign agent. In particular, the administering may be through inhalation, oral injection, surgical injection, rectal absorption, intravenous, subcutaneous or intramuscular means.

### BRIEF DESCRIPTION OF THE FIGURES

FIG. 1 shows a 2-dimensional TEM image (a) and 3-dimensional mathematical model (b) of the same cubic membrane organization observed in the inner mitochondrial membrane of 10 days starved amoeba Chaos carolinense. The bar shown in (a) represents 500 nm

FIG. 2 shows the images that result from an analysis carried out with two colour fluorescence. Green fluorescence-tagged oligonucleotides (ODNs) (a) were incubated with amoeba Chaos cubic mitochondria labelled with MitoTracker Red (b) for an hour at room temperature. Overlay of the 2 images shows co-localization of the fluorescent ODN and Chaos cubic mitochondria, suggesting mitochondrial uptake of ODN (c). The Green fluorescence-tagged ODNs, red labelled amoeba Chaos cubic mitochondria and the results of the co-localization of both are shown as bright dots in (a), (b) and (c) respectively. Some of the corresponding spots found in (a), (b) and (c) are highlighted with black arrows. The results prove that mitochondria with membranes of cubic structure are able to uptake fluorescence-tagged ODNs. The results illustrate a representative experiment of at least three others with similar findings.

FIG. 3 shows TEM images of mitochondria comprising membrane(s) of cubic structure isolated from 10 days starved Chaos cells before (a) and after (b) incubation with ODNs. Mitochondria with membranes of cubic organization interact significantly with ODN molecules (b) but not with the same amount of ‘non-cubic’ mitochondria isolated from well-fed amoeba (c) or mouse liver (d) which act as controls. It seems the multiple pores (shown in (a) and (b) with arrows) at the surface of cubic mitochondria play an important role in facilitating passive uptake of ODNs.

FIG. 4 shows a low (A) and high (B) magnification of TEM images of cubic membranes in the mitochondria isolated from 10 days starved Chaos cells. Mitochondrial membrane of cubic structure interacts significantly with ODNs (C). Multiple electron-dense intra-mitochondrial inclusions may represent cubic membrane-mediated ODNs interaction (D). However, as a result of this interaction, the mitochondrial inner membrane lost its cubic architecture and no organized membrane morphology could be detected. The results show that mitochondria with membranes of cubic structure have high affinity to “concentrate” ODNs within the complex and convoluted channels of cubic structure.

FIG. 5 shows the results of a gel retardation study. Same amount of mitochondrial proteins (26 μg/μl) from amoeba cubic mitochondria and mouse liver mitochondria were incubated with the same amount of ODN (0.1 μg/μl) molecules. The mitochondria with cubic membrane organization are able to retard ODNs mobilization (lane 2) towards the positive pole as compared to the same amount of pure ODNs (lane 1) and ODNs incubated with isolated mice liver mitochondria (lane 3). This proves that mitochondria with membranes of cubic structure retain ODN molecules against electrical field dissociation. The results illustrate a representative experiment of at least three others with similar findings, showing one of duplicate samples.

FIG. 6 shows the results of an in vitro study of ODN-cubic membrane complex internalization into MCF-7 cells (human breast adenocarcinoma cell line). MCF-7 cells were incubated with stable submicron sized particles of cubic membranes containing ODNs (“ODN-delivery particle”). After 5 hours of treatment, highly fluorescent MCF-7 cells were observed under low (a), high (b) magnification by fluorescence and (c) phase-contrast microscopy. The green fluorescence-tagged ODNs, observed in the cytoplasm and the nucleus (a and c: 64×, b: 200×) are shown as bright spots in contrast to the grey background in (a) to (c). The results show that the tagged-ODNs were located in both the nucleus and cytoplasm of the MCF-7 cells. The results illustrate a representative experiment of at least three to five others with similar findings.

FIG. 7 shows TEM images of the MCF-7 cells 5 hr post-transfection with ODN-delivery particle(s). There are no inclusion bodies observed in MCF-7 cells without ODNs treatment (a) or MCF-7 cells incubated with naked ODNs (b). However, multiple inclusion bodies were observed in those MCF-7 cells that have been transfected with ODN-delivery particle(s) for 5 h (c and d). Multiple ‘electron-dense’ inclusion bodies were observed in the cytoplasm and some targeted the nucleus (N). At higher magnification, membrane association was frequently observed between electron-dense inclusion bodies and nuclear membranes (e and f).

FIGS. 8A and B show the results of a mass spectrum on whole Chaos cell lipids. The overall lipid profiles of both fed (A) and 7 days starved (B) samples were the same except for the alterations of the amounts. Three major species (748, 776 and 957 m/z) in the range 600-1000 m/z are highlighted in the lipid profiles of both samples.

FIG. 9 shows the results when three major phospholipids, PC (38:5), PE (38:5) and PI (40:10), were characterized in the lipid profiles of both fed vs. 7 days starved amoeba Chaos samples. The corresponding chemical structure of each lipid class was determined and is displayed in FIG. 9. There is a significant increase in plasmalogen PC (22:5/16:0), and a decrease in both plasmalogen PE (22:5/16:0) and diacyl PI (22:5/22:5) upon amoeba starvation i.e. cubic structure formation. These three compounds may thus play an essential role in the formation of membranes with cubic structure.

FIG. 10 shows the relative distribution of fatty acids of fed and 7 days starved Chaos cells. As can be seen in FIG. 10, linoleic acid (C18:2) was significantly decreased in the 7 days starved Chaos cells compared to the fed Chaos cells. There was also a significant increase in docosapentaenoic acid (C22:5) in total amoeba lipid extracts, upon 7 days of starvation compared to the C22:5 fatty acids of the fed Chaos cells. It was clearly demonstrated that Chaos cells responded to starvation by having more highly polyunsaturated fatty acids in their membrane lipids. The highly polyunsaturated fatty acid content in starved amoeba may partly explain the ability for mitochondrial cubic membranes transformation under starvation stress condition.

FIG. 11 shows TEM images of liposomal construction of total lipid extracted from fed and 7 days starved Chaos cells. Liposomes prepared from the lipids extracted from fed amoeba tended to form multi-lamellar (A) or sponge membrane structures (B); while the liposomes made from lipids extracted from 7 days starved Chaos cells displayed cubic (C) or hexagonal (D) organization. Fast Fourier transformation (FFT) analysis confirmed symmetrical organizations (displayed in lower right of panel of C and D). The results show that lipids play a major role in biological membrane phase transition in Chaos mitochondria.

FIG. 12 shows mitochondrial protein profiles of amoeba Chaos cells (fed vs. 7 days starved) by Automated Electrophoresis System (Experion™). The results of the protein profiling confirm the results of Example 6, which reveal that membrane-bound proteins are not the major players of membrane transformation.

FIG. 13 shows the results of a TEM study on the effects of continuous feeding of docosapentaenoic acids (DPA, 22:5) to amoeba Chaos cells with the presence of their food organisms Paramecium. (A) Fed, (B) 7 days starved (C) control Chaos cells with continuous feeding of DPA (n-3) for 5 days; and continuous feeding DPA (n-6) (D) to amoeba Chaos cells for 5 days. A continuous feeding of DPA (n-6) to fed Chaos cells enhanced their inner mitochondrial membrane transformation from random tubular (A) to cubic organization (D); while feeding DPA (n-3) (C) did not have the same drastic effects on their inner mitochondrial membrane organization. These observations strongly suggest the key role of DPA (n-6) but not (n-3) in the cubic structure formation of the membranes in the mitochondria of amoeba Chaos cells.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

### DEFINITIONS

For convenience, certain terms employed in the specification, examples and appended claims are collected here.

The term “agent delivery particle” is herein defined as a small component that comprises at least one membrane and at least one foreign agent. It may be the result of fragmentation of an isolated organelle comprising at least one membrane of cubic structure and at least one foreign agent. An example of an agent delivery particle is the ODN-delivery particle used in Example 4 which is a result of fragmentation by way of sonication of mitochondria isolated from 7 days starved amoeba comprising at least one membrane of cubic structure and ODN as the foreign agent. However, the agent delivery particle may be prepared according to any method known to a skilled person.

The term “biological membrane” is herein defined as an enclosing or separating amphipathic layer of biological or natural origin that acts as a barrier within or around a cell or an organelle. It is, almost invariably, a lipid bilayer, composed of a double layer of lipid-class molecules, specifically phospholipids, with occasional proteins intertwined, some of which function as channels. The biological membrane may or may not have a cubic structure.

The term “cell” used interchangeably here with “target cell” is herein defined as any cell that is from at least one organ such as a heart, blood vessel, lungs, liver, kidney, skin, cornea or the cell is from at least one non-organ such as bone marrow cell, stem cell or gamete and the like.

The term “Chaos carolinense” used interchangeably here with “Chaos cells” is herein defined as a genus of giant amoebae, varying from 1-5 mm in length. They are closely related to Amoeba, but have several hundred nuclei, while Amoeba only has one. Chaos move by pseudopodia. They do not have a hard cell wall. The cytoplasm is divided into the endoplasm which is fluid and contains the many nuclei, granules, and food vacuoles, and the ectoplasm which is more viscous and does not contain any granules.

The term “fed” is herein defined as the state the cells, for example, but not limited to, Chaos cells reach when they are cultured according to the protocol for the continuous simple mass culture of amoeba feeding on only one food organism Paramecium multimicronucleatum described in Tan et al., 2005.

The term “liposome” is herein defined as a spherical vesicle composed of a bilayer lipid membrane of cubic structure which is of a phospholipid and cholesterol bilayer. Liposomes can be composed of naturally-derived phospholipids with mixed acyl chains (like egg phosphatidylcholine), or of pure surfactant components like DOPE (dioleoylphosphatidylethanolamine). Liposomes usually contain a core of aqueous solution. Accordingly, the “liposome” comprising a cubic structure according to the present invention are distinct from the liposome of the prior art which do not comprise membranes of cubic structures.

The term “lipid membrane with cubic structure” is herein defined as a membrane prepared substantially from lipids that self-assemble in at least one aqueous medium to form cubic structure. The lipids may be extracted from at least one membrane of cubic structure or obtained from a commercial supplier. For example the liposome in Example 6 has a lipid membrane with cubic structure when the lipids extracted from the mitochondria with membrane of cubic structure self-assembled into lipid membranes of cubic structure when in contact with water. The lipid may be at least one lipid selected from the group consisting of plasmalogen phosphatidylcholine (PC), plasmalogen phosphatidylethanolamine (PE) and diacyl phosphatidylinositol (PI).

The term “membrane with cubic structure” is used interchangeably here with “cubic membrane” and “cubic membrane organization”. The term is herein defined as three-dimensional (3d) nano-periodic structures that occur in a wide variety of living systems. They are based on highly curved surfaces that are mathematically analogous to triply periodic minimal surfaces used in describing both crystalline and liquid crystalline materials at a variety of length scales. A membrane may be considered to have a cubic structure if the relative amount of C22:5 fatty acid increases by 50-80% compared to a membrane of non-cubic structure. In particular, the relative amount of C22:5 fatty acid increase may be about 60%. More in particular, the relative amount of C22:5 fatty acid increase may be about 70%. A membrane may be considered to have a cubic structure if the relative amount of plasmalogen phosphatidylcholine (PC) may increase by about at least 80% compared to a membrane of non-cubic structure. In particular, the relative amount of PC may increase by about at least 100%. More in particular, the relative amount of PC may increase by about at least 122%. A membrane may be considered to have a cubic structure if the relative amount of plasmalogen phosphatidylethanolamine (PE) may decrease by about at least 10% compared to a membrane of non-cubic structure. In particular, the relative amount of PE may increase by about at least 15%. More in particular, the relative amount of PE may increase by about at least 20%. A membrane may be considered to have a cubic structure if the relative amount of diacyl phosphatidylinositol (PI) may decrease by about at least 10% compared to a membrane of non-cubic structure. In particular, the relative amount of PI may increase by about at least 20%. More in particular, the relative amount of PI may increase by about at least 35%.

The term “organelle” is herein defined as a specialized subunit within a cell that has a specific function, and is typically separately enclosed within its own lipid membrane. Examples of organelles are chloroplast, cytoskeleton, Golgi apparatus, mitochondrion, nucleus, rough endoplasmic reticulum (RER), smooth endoplasmic reticulum (SER), vacuole, vesicle, autophagosome, glyoxysome, hydrogenosome, lysosome, peroxisome, and the like. Examples of non-membrane bound organelles are centriole, ribosome, cytoskeleton, myofibrils, cilia, and the like.

The term “starved” is herein defined as the state the cells, for example, but not limited to, Chaos cells, reach when they are not cultured according to the standard protocol(s) for the continuous culture. In particular, with reference to Chaos cells, it is the state the Chaos cells reach when they are not cultured according to the protocol for the continuous simple mass culture of amoeba feeding on only one food organism Paramecium multimicronucleatum that has been described previously (Tan et al., 2005). A state of starvation may be reached by the cells, for example by Chaos cells, by not feeding them for one to 12 days. In particular, the state of starvation may be reached by not feeding them for at least 10 days. More in particular, the state of starvation may be reached by the cells by not feeding them for at least 7 days.

The term “subject” is herein defined as a mammal including a human in need of therapy for, or susceptible to, a condition or its sequelae. The subject may include dogs, cats, pigs, cows, sheep, goats, horses, rats, mice and humans. The term “subject” does not exclude an individual that is normal in all respects.

A person skilled in the art will appreciate that the present invention may be practised without undue experimentation according to the method given herein. The methods, techniques and chemicals are as described in the references given or from protocols in standard biotechnology and molecular biology text books.

In one aspect of the present invention there is provided at least one agent delivery particle comprising at least one biological membrane and at least one foreign agent.

The foreign agent of the agent delivery particle may be selected from the group consisting of amino acids, anabolics, analgetics and antagonists, anaesthetics, anthelmintic, anti-adrenergic agents, anti-asthmatics, anti-atherosclerotics, antibacterials, anticholesterolics, anti-coagulants, antidiarrheal, antidepressants, antidotes, anti-emetics, anti-epileptic drugs, anti-fibrinolytics, anti-inflammatory agents, antihypertensives, antimetabolites, antimigraine agents, antimycotics, antinauseants. antineoplastics, anti-obesity agents, antiprotozoals, antipsychotics, antirheumatics, antiseptics, antivertigo agents, antivirals, appetite stimulants, bacterial vaccines, bioflavonoids, calcium channel blockers, capillary stabilizing agents, coagulants, corticosteroids, detoxifying agents for cytostatic treatment, diagnostic agents (like contrast media, radiopaque agents and radioisotopes), electrolytes, enzymes, enzyme inhibitors, ferments, ferment inhibitors, gangliosides and ganglioside derivatives, hemostatics, hormones, hormone antagonists, hypnotics, immunomodulators, immunostimulants, immunosuppressants, minerals, muscle relaxants, neuromodulators, neurotransmitters and neurotrophins, osmotic diuretics, parasympatholytics, parasympathomimetics, peptides, polysaccharides, proteins, psychostimulants, respiratory stimulants, sedatives, serum lipid reducing agents, smooth muscle relaxants, sympatholytics, sympathomimetics, vasodilators, vasoprotectives, vectors for gene therapy, viral vaccines, viruses, vitamins, oligonucleotides and derivatives, saccharides, polysaccharides, glycoproteins, hyaluronic acid, and any excipient that can be used to stabilize a proteinaceous therapeutic.

In particular, the foreign agent may be selected from the group consisting of hydrophilic drug, hydrophobic drug, nucleic acid, polypeptide, polysaccharide, virus, and vitamin. More in particular, the foreign agent may be nucleic acid. The nucleic acid may be deoxyribonucleic acid (DNA), ribonucleic acid (RNA), siRNA or artificial nucleic acids which include peptide nucleic acid (PNA), Morpholino and locked nucleic acid (LNA), as well as glycol nucleic acid (GNA) and threose nucleic acid (TNA).

The biological membrane may be any membrane with cubic structure that surrounds cells or organelles or may be any membrane derivable from a membrane with cubic structure. Examples include but are not limited to intracellular membranes of the mitochondria or endoplasmic reticulum, plasma membranes of animal and plant cells, T-tubules in denervated skeletal muscle cells and the like. More in particular, the membrane may be extracted from the cell and/or organelle and brought in contact with the foreign agent.

The agent delivery particle may be obtained from at least one organelle comprising at least one membrane and at least one foreign agent. The organelle may be selected from the group consisting of chloroplast, Golgi apparatus, mitochondrion, nucleus, rough endoplasmic reticulum (RER), smooth endoplasmic reticulum (SER), vacuole, and vesicle. In particular, the organelle may be mitochondrion. More in particular, the organelle may comprise at least one membrane of cubic structure prior to contact with the foreign agent.

The agent delivery particles of the present invention comprising membranes derived from membranes of cubic structure provide a means for efficient and passive uptake and release of foreign agent(s) without the electrostatic effect induced by cationic lipids of Cubosomes® and liposomes described in the prior art thus making it a safe means of delivery of foreign agent(s) to target cells.

The agent delivery particle may be 100 to 600 nm in diameter. In particular, the agent delivery particle may be 200 to 500 nm in diameter. More in particular the agent delivery particle may be 250 to 400 nm in diameter.

In particular, the agent delivery particle may be a result of fragmentation of the organelle. Fragmentation may be carried out using mechanical or chemical methods known in the art. In particular, the method of fragmentation may be at least one form of physical attrition using at least one means of puncturing the organelle using a needle, microinjection and the like. The method of fragmentation may be other forms of physical attrition such as applying current, voltage, electromagnetic waves, ultrasound waves and the like to the organelle of the present invention. More in particular, the method of fragmentation may be sonication using ultrasound waves.

According to another aspect, the present invention also provides at least one agent delivery particle comprising at least one biological membrane of cubic structure and at least one foreign agent. In particular, the biological membrane may be any membrane with cubic structure that surrounds cells or organelles or may be any membrane derivable from a membrane with cubic structure. Examples include but are not limited to intracellular membranes of the mitochondria or endoplasmic reticulum, plasma membranes of animal and plant cells, T-tubules in denervated skeletal muscle cells and the like. More in particular, the membrane may be extracted from the cell and/or organelle and brought in contact with the foreign agent.

The agent delivery particle according to any aspect of the present invention may be for use in medicine. In particular, the agent delivery particle of the present invention may be for use in gene therapy.

According to another aspect, the present invention provides at least one method of preparing at least one agent delivery particle, comprising the steps of:
(a) isolating at least one organelle from at least one cell;
(b) contacting the organelle with at least one foreign agent; and
(c) preparing at least one agent delivery particle from the organelle comprising the foreign agent of step (b), wherein the obtained agent delivery particle comprises at least one biological membrane and the foreign agent.

In particular, the method of isolating the organelle of step (a) may be through the use of subcellular fractionation and analysis kits commercially available in Invitrogen, Sigma-Aldrich and the like or it may be through methods such as free-flow electrophoresis and/or differential centrifugation. More in particular, the organelle isolated in step (a) may comprise at least one membrane of cubic structure.

In particular, the method of contacting the organelle with the foreign agent of step (b) may be through incubating the organelle in at least one medium comprising the foreign agent in suitable conditions for the uptake of the foreign agent into the organelle. The method of contacting the organelle with the foreign agent may be through injection or active means such as by applying current, voltage or electromagnetic waves to the organelle, or by chemical dialysis and the like. More in particular, the method of contacting the organelle with the foreign agent may be through incubating the organelle in at least one culture medium (e.g. DMEM) comprising the foreign agent in the incubator with 5% CO₂ at 37° C. for 5 h.

The foreign agent may be selected from the group described above. More in particular, the foreign agent may be selected from the group consisting of hydrophilic drug, hydrophobic drug, nucleic acid, polypeptide, polysaccharide, virus, and vitamin. In particular, the foreign agent may be nucleic acid.

In particular, the agent delivery particle of step (c) may be prepared by fragmenting the organelle comprising the foreign agent of step (b). Fragmentation may be performed according to any one of the methods described above.

According to another aspect, the present invention provides at least one isolated organelle comprising at least one membrane and at least one foreign agent. In particular, the isolated organelle may comprise at least one membrane of cubic structure prior to contact with the foreign agent. The isolated organelle may be selected from the group consisting of chloroplast, Golgi apparatus, mitochondrion, nucleus, rough endoplasmic reticulum (RER), smooth endoplasmic reticulum (SER), vacuole, and vesicle. In particular, the isolated organelle may be mitochondrion.

The isolated organelle of the present invention may be isolated from any organism. In particular, the organism may be monera, protista, fungi, plants or animals. More in particular, the organism may be unicellular amoeba. The amoeba may be Chaos carolinense.

The membrane(s) of the isolated organelle may achieve cubic structure when they encounter stressful conditions. Examples of the stressful conditions may be starvation, light deprivation, over exposure to light, over exposure to UV, protein over-production, virus-infections, oxidative stress, reduced cell volume and the like.

In particular, the foreign agent may be selected from the group described above.

According to a further aspect, the present invention provides at least one method of preparing at least one organelle comprising at least one membrane of cubic structure by contacting at least one cell comprising the organelle with fatty acid C22:5. In particular, the fatty acid C22:5 may be all-cis-7,10,13,16,19-docosapentaenoic acid, a n-3 fatty acid or the fatty acid may be all-cis-4,7,10,13,16-docosapentaenoic acid, a n-6 fatty acid. The n-3 fatty acid C22:5 with the trivial name clupanodonic acid, may be commonly called DPA or it may be an intermediary between eicosapentaenoic acid (EPA, 20:5 n-3) and docosahexaenoic acid (DHA, 22:6 n-3). The. The n-6 fatty acid C22:5 with the trivial name Osbond acid, may be formed by an elongation and desaturation of arachidonic acid 20:4ω-6.

In particular, the method of contacting the cell with the fatty acid C22:5 may be through incubating the cell in at least one medium comprising the fatty acid C22:5 in suitable conditions for the uptake of the fatty acid C22:5 into the cell. The method of contacting the cell with the fatty acid C22:5 may be through injection or active means such as by applying current, voltage or electromagnetic waves to the cell, or by chemical dialysis and the like. More in particular, the method of contacting the cell with the fatty acid C22:5 may be through incubating the cell in at least one culture medium (e.g. DMEM) comprising the fatty acid C22:5 at a final concentration of about 0.05 to 1 mM for at least 5 days. The final concentration of fatty acid C22:5 may be 0.1 mM to 0.5 mM. In particular, the final concentration of fatty acid C22:5 may be about 0.1 mM.

According to a further aspect, the present invention provides at least one method of preparing at least one organelle comprising at least one foreign agent, comprising the steps of:
(a) isolating at least one organelle from at least one cell; and
(b) contacting the organelle with at least one foreign agent.

In particular, the isolated organelle of step (a) may comprise at least one membrane of cubic structure prior to contacting with the foreign agent. In particular, the isolated organelle may be selected from the group consisting of chloroplast, Golgi apparatus, mitochondrion, nucleus, rough endoplasmic reticulum (RER), smooth endoplasmic reticulum (SER), vacuole, and vesicle. More in particular, the isolated organelle may be mitochondrion. The isolated organelle of step (a) may be isolated from any suitable source, for example from at least one amoeba. In particular, the amoeba may be Chaos carolinense.

The method of isolating and contacting the organelle may be performed according to any one of the methods described above.

According to a further aspect, the present invention provides at least one liposome comprising at least one lipid membrane of cubic structure. In particular, the liposomes may be small unilamellar vesicles or large unilamellar vesicles containing a single lipid bilayer and/or large multilamellar vesicles, containing multiple bilayers (onion-like in structure) with an aqueous space separating each bilayer from the other. The liposomes of the present invention may also be multivesicular vesicles. The liposomes may have at least one core of aqueous solution or contain no aqueous material at all.

More in particular, the liposome of the present invention may be made from at least one lipid of at least one organelle comprising at least one membrane of cubic structure. The lipids of the liposome of the present invention may be from the organelle selected from the group consisting of chloroplast, Golgi apparatus, mitochondrion, nucleus, rough endoplasmic reticulum (RER), smooth endoplasmic reticulum (SER), vacuole, and vesicle. In particular, the lipids of the liposome of the present invention may be phospholipids. More in particular, the phospholipids may be selected from the group consisting of phosphatidylcholine (PC) phosphatidylethanolamine (PE) and/or phosphatidylinositol (PI).

The phospholipids PC, PE and/or PI may be naturally occurring. In particular, the phospholipids may be extracted from at least one cell. More in particular, the phospholipids may be extracted from at least one membrane of the cell. Even more in particular, the phospholipids may be extracted from at least one membrane of cubic structure of the cell. The phospholipids may be commercially synthesised.

According to another aspect, the liposome according to the invention may comprise at least one lipid selected from the group consisting of plasmalogen phosphatidylcholine (PC), plasmalogen phosphatidylethanolamine (PE) and diacyl phosphatidylinositol (PI).

The liposome of the present invention may comprise at least one foreign agent. The foreign agent may be selected from the group described above. In particular, the liposome of the present invention may be for use in delivery of the foreign agent into at least one cell in vivo or in vitro. The delivery of the foreign agent into the cell may be invasive or non-invasive. In particular, the delivery of the foreign agent into the cell in vivo may be through oral, nasal, pneumonial and rectal routes and/or through injection. In particular, the delivery of the foreign agent into the cell in vitro may be through transfection, injection and the like.

The liposome of the present invention may be for use in medicine. In particular, the liposome of the present invention may be for use in gene therapy.

According to a further aspect, the present invention provides at least one method of preparing the liposome of the present invention comprising the steps of:
(a) isolating at least one organelle comprising at least one membrane of cubic structure;
(b) isolating lipids from the membrane; and
(c) preparing at least one liposome with at least one membrane of cubic structure by contacting the lipids of step (b) with at least one aqueous medium.

According to another aspect, the present invention provides at least one method of preparing the liposome of the present invention by contacting at least one lipid selected from the group consisting of plasmalogen phosphatidylcholine (PC), plasmalogen phosphatidylethanolamine (PE) and diacyl phosphatidylinositol (PI) with at least one aqueous medium.

Isolating the organelle may be carried out using any of the isolation methods described above. The lipids may be isolated from the membrane using the methods taught in Folch (Folch et al., 1957), and/or Bligh and Dyer (Bligh and Dyer, 1959).

In particular, the aqueous medium may be any medium that comprises primarily of water. The aqueous medium may be water (which term is to be understood as covering any aqueous liquid containing buffers, salts or dissolved compounds), glycerol, propylene glycol, a polyethylene glycol, or a mixture of two or more thereof. Typically, the proportion of the lipid component to the aqueous medium in the composition will be within the range of from 1:1 to 1:40, in particular from 1:2 to 1:20 and more in particular from 1:4 to 1:10. More in particular, the aqueous medium may be miniQ water.

According to another aspect, the present invention provides at least one method of delivery of at least one foreign agent comprising either contacting the agent delivery particle or the liposome of the present invention with at least one cell in vivo or in vitro.

In particular, the foreign agent and the means of contacting may be selected from the foreign agents and the methods described above.

According to another aspect, the present invention provides at least one use of the organelle of the present invention for preparing at least one agent delivery particle for treating at least one subject. In particular, the subject may be at least one human in need of therapy for, or susceptible to, at least one condition or sequelae of the condition.

According to another aspect, the present invention provides at least one use of the liposome of the present invention for preparing at least one medicament for treating at least one subject. In particular, the subject may be at least one human in need of therapy for, or susceptible to, at least one condition or sequelae of the condition.

In particular, the medicament may be in the form of pills, capsules, solutions, ointments, creams and the like.

According to still another aspect, the present invention provides at least one kit for delivery of at least one foreign agent, the kit comprising the organelle, the agent delivery particle, and/or the liposome of the present invention. In particular, the kit may further comprise instructions on how to use the kit.

According to a further aspect, the present invention provides at least one method of detecting and/or monitoring the presence of at least one pathology in at least one cell, comprising detecting an increase of cubic ER membrane compared to a control, the increase correlating to the presence of the pathology.

According to another aspect, the present invention provides at least one method of gene therapy comprising administering at least one subject with the agent delivery particle of the present invention comprising nucleic acid as the foreign agent, or the liposome of the present invention comprising nucleic acid as the foreign agent. In particular, the administering may be through inhalation, oral injection, surgical injection, rectal absorption, intravenous, subcutaneous or intramuscular means.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Spring Harbor Laboratory, New York (2001).

### Example 1

### Cell Cultures

The mixed culture of the amoeba, Chaos carolinense “Chaos cells”, used in this study was originally obtained from Carolina Biological Supply Co. (Burlington, N.C.). A protocol for the continuous simple mass culture of amoeba feeding on only one food organism Paramecium multimicronucleatum has been described previously (Tan et al., 2005).

Human breast cancer MCF-7 cells (human breast adenocarcinoma cell line) obtained from American Type Culture Collection (ATCC Cat. No: HTB-22) were grown in Dulbecco's Modified Eagle's Medium (DMEM) supplemented with 2 mM glutamine, 10% Fetal Bovine Serum and 10% antibiotics (penicillin/streptomycin) in the incubator with 5% CO₂ at 37° C.

### Amoeba Chaos Cells Harvest

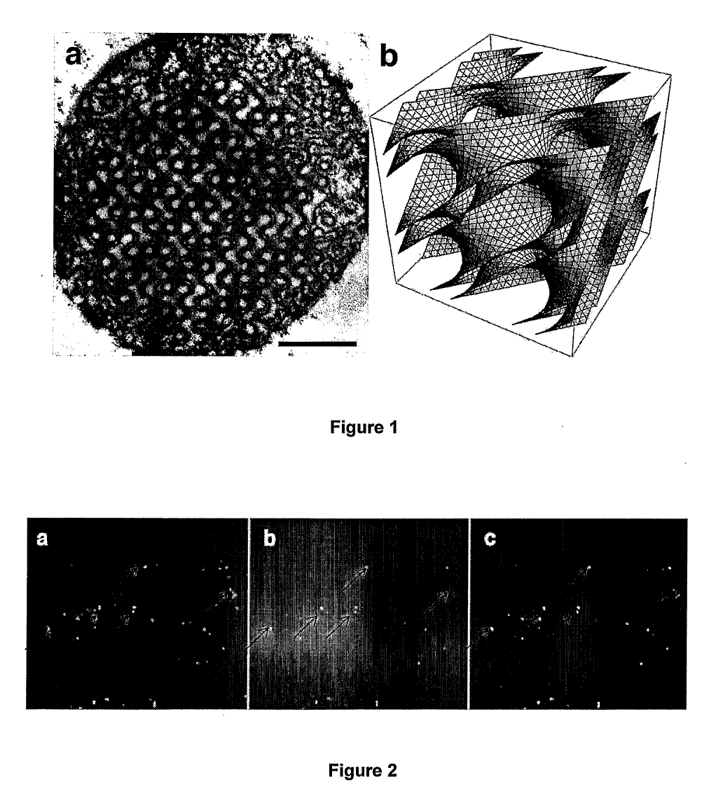

Prior to harvesting the Chaos cells, any impurities and P. multimicronucleatum were gently removed by washing the culture several times with amoeba inorganic medium containing 0.5 mM CaCl₂, 0.05 mM MgSO₄, 0.16 mM K₂HPO₄, 0.11 mM KH2PO4, made up in MilliQ water, at a depth of 7-10 mm and kept in the dark, at 22° C.-24° C. on bench top or in a 22° C. cool incubator (Sanyo MIR-553) as described in Tan et al., 2005. The Chaos cells were dislodged from the bottom of the culture dishes by squirting medium with a washing bottle and Chaos cells were collected into tall beakers (500 ml) containing amoeba inorganic medium and let settle by sedimentation. The supernatant was discarded and the clean amoeba Chaos culture was ready for use. No food organisms were added to Chaos culture for 10 days in the starvation study. Chaos cells were starved for 10 days to ensure complete transformation of the inner mitochondrial membrane to the membrane of cubic structure (Deng and Mieczkowski, 1998) as shown in FIG. 1a.

Isolation of Mitochondria from Amoeba Chaos

Fed and starved Chaos cells were washed thoroughly to remove all the impurities and were gently hand-homogenized at 4° C. in the ice cold isolation buffer (10 mM EDTA, 250 mM sucrose, 100 mM KCl, 50 mM Tris HCl (pH 7.4), 1 mM K₂HPO₄, 2% Bovine Serum Albumin (BSA)). The crude cell homogenate was centrifuged at 1,500 rpm for 4 min and the first pellet (containing many well-preserved nuclei, few mitochondria and some broken fragments of plasmalemma) was discarded. The supernatant was transferred to a new tube and centrifuged at 4,500 rpm for 10 min at 4° C. The second pellet containing mitochondria was resuspended in 0.2% BSA isolation medium (same composition as the isolation buffer except the varied concentration of BSA) for further use. Bradford's method was used to quantify the total mitochondrial proteins in the isolated sample (Bradford, 1976). The differential centrifugation procedures were carried out at 4° C. in a Jouan Centrifuge (BR4i).

Isolation of Liver Mitochondria from Mice

Mouse liver mitochondria were isolated by the differential centrifugation as previously described with modifications (Almsherqi et al., 2006). Briefly, following asphyxiation by CO₂, the tissues were quickly removed and placed in chilled (4° C.) isolation medium A (100 mM sucrose, 100 mM KCl, 50 mM Tris-HCl (pH 7.4) 1 mM K₂HPO₄, 50 μM EGTA and 0.2% BSA). The tissue was repeatedly washed with isolation medium A to remove any adhering fats and blood. Any visible connective tissues were removed with a scalpel. Thereafter, the tissue was homogenized in 50 ml isolation medium A with a Teflon glass homogenizer at 10,000 rpm with five strokes. The homogenate was immediately centrifuged at 2,300 rpm for 10 min. The supernatant was transferred into a new tube and centrifuged at 8,900 rpm for 10 min. The pellet was washed and resuspended in 50 ml medium A, and recentrifuged at 6,900 rpm for 10 min. Following every centrifugation step, remnants of fat on the wall and brim of the centrifuge tubes were removed with rolled filter papers. The final pellet was washed and suspended in chilled medium B (225 mM mannitol, 75 mM sucrose, 10 mM Tris-HCl (pH 7.4) and 100 μM EDTA). The differential centrifugation procedures were carried out at 4° C. in a Jouan Centrifuge (BR4i).

Co-Localization of Mitochondria with Membranes of Cubic Structure and Tagged-ODNs

Fluorescein-tagged phosphorothioate oligonucleotides 18 mer (PS-ODNs) (5′ tct ccc agc gtg cgc cat 3′) (Sigma-Proligo) was used in this study. Mitochondria with membrane(s) of cubic structure (“Cubic mitochondria”) isolated from 10 days starved Chaos cells were first resuspended in the 0.2% isolation medium according to the procedure described in the section on mitochondria isolation above. The mitochondria were then incubated with 10 nM MitoTracker (Red) CMXRos (Molecular Probes), a mitochondria-selective fluorescent probe, for 15 min at room temperature. Next, the mitochondria sample was incubated with 1.2 μg/μl fluorescein-tagged ODNs, as described in the section above, at 1:1 volume ratio for 1 h at room temperature. The sample was then purified by centrifugation in an Eppendorf centrifuge 5412C at 4,500 rpm for 10 min and washed 3 times with amoeba inorganic medium (described in the section on Chaos cell harvest) before fluorescence microscopy examination.

An equivalent amount of ‘non-cubic’ mitochondria (isolated either from fed Chaos cells or mice (C57BL/6J) liver) and ODNs were incubated for comparison. All samples were observed under a fluorescence microscope (Zeiss Axioplan2 imaging, Metasystem imaging software) using two different wavelengths (green excitation wavelength: 520 nm, emission wavelength: 495 nm and red excitation wavelength: 579 nm, emission wavelength: 599 nm) for the co-localization study.

Wavelength-tuned fluorescence microscopy was used to detect the locations of both mitochondria and ODNs. In the usual (non-cubic) mitochondria, the locations of ODNs and mitochondria were distinct. In contrast, preparations from those starved Chaos cells with cubic mitochondrial membranes revealed coincidence in the locations of ODNs (FIG. 2a, shown as bright spots in contrast to the grey background) and mitochondria (FIG. 2b, shown as bright spots in contrast to the grey background); for example, simultaneous excitation of both red and green fluorescent probes gave a visible yellow signal (FIG. 2c, shown as bright spots in contrast to the grey background), indicative of ODNs location within the mitochondria. Some of the corresponding spots found in FIGS. 2(a), (b) and (c) are highlighted with black arrows. The results prove that mitochondria with membranes of cubic structure are able to uptake fluorescence-tagged ODNs. The results illustrate a representative experiment of at least three others with similar findings.

### Example 2

### Transmission Electron Microscopic (TEM) Study

Isolated mitochondria pellets (from fed, 10 days starved Chaos cells and mice liver) from Example 1 and transfected MCF-7 cell pellet prepared according to Example 4 were studied by the conventional TEM technique. Samples in the pellet forms were first fixed individually with 2.5% glutaraldehyde in 1×PBS at 4° C. After washing 4 times with 1×PBS buffer over 1 h to remove glutaraldehyde, the samples were post-fixed with 1% osmium tetroxide in 1× PBS buffer (pH 7.4) for 20 min at room temperature. The specimens were then washed 4 times with the 1×PBS buffer at 4° C. Thereafter 3% gelatin in PBS was added for 30 min before spinning down to collect the pellets at 2,500 rpm for 5 min. The specimens were stored at 4° C. overnight and the steps of adding 2.5% glutaraldehyde in PBS and washing with PBS were repeated for another cycle. The bigger specimens were trimmed into small cubes (1 mm in length) and washed with distilled water for 5 min. Dehydration through ethanol series was done using ethanol of 25%, 50%, 75%, 95% and 100% for 10 min each at room temperature. The specimens were then infiltrated with 100% acetone: resin in 1:1 ratio for 30 min at room temperature and with 100% acetone: resin in 1:6 ratio overnight. Ultrathin (70-90 nm) sections of specimens were prepared with a diamond knife and collected on 300-mesh copper grids. Sections were then stained with lead citrate for 10 min. Ultrathin sections were viewed on a transmission electron microscope (JEOL 100 CX II) at 80 kV.

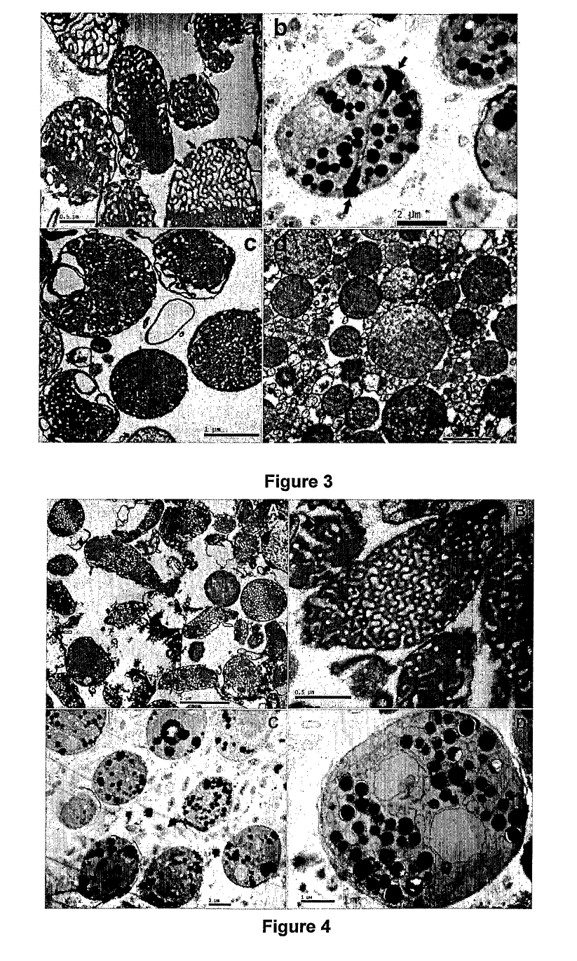

TEM images substantiate the apparent colocalization in Example 1 of ODNs and cubic mitochondria. Mitochondria comprising membrane(s) of cubic structure (“cubic mitochondria”) isolated from 10 days starved Chaos cells before and after incubation with ODNs are shown n FIGS. 3(a) and (b) respectively. Cubic mitochondria interact significantly with ODN molecules (b) but not with the same amount of ‘non-cubic’ mitochondria isolated from well-fed amoeba (c) or mice liver (d) which act as controls. Multiple electron-dense intra-mitochondrial inclusions may represent cubic membrane-mediated ODNs interaction. The multiple pores (shown in FIGS. 3(a) and (b) with black arrows) at the surface of cubic mitochondria play an important role in facilitating passive uptake of ODNs. These surface pores, are both abundant and very uniform in diameter in cubic mitochondria.

A low and high magnification of TEM images of cubic mitochondria isolated from 10 days starved Chaos cells shown in FIGS. 4(A) and (B) respectively reveal that cubic mitochondria interact significantly with ODNs. This is especially seen in FIG. 4(C) where multiple intra-mitochondrial inclusions were formed, most likely due to association between ODNs and the internal membrane lipids in starved cubic mitochondria. Moreover, as a result of this association, the characteristic morphological signature of the cubic membranes within the mitochondria was lost and no organized membrane morphology could be detected (FIG. 4D). The results show that mitochondria with membranes of cubic structure have high affinity to “concentrate” ODNs within the complex and convoluted channels of cubic structure.

Morphometric calculations suggest that 20-40% of the total volume of the mitochondria was occupied by the inclusion bodies and about 90% of all the cubic mitochondria showed an association with ODNs.

### Example 3

### Gel Retardation Assay

To further examine the ability of cubic mitochondria to retain ODNs molecules, gel retardation tests were performed using mice liver mitochondria and amoeba mitochondria subjected to starvation for 10 days, both incubated with the same amount of ODNs. Since the formation of ODNs-cubic membrane complexes is expected to retard electrophoretic mobility of ODN component in the gel matrix, this assay tests the degree of association between ODNs and mitochondria.

The quantity of mitochondrial proteins was determined by the Bradford method (Bradford, 1976). An equal amount of mitochondria (260 μg mitochondria proteins) from 10 days starved amoeba and mouse liver were incubated separately with 0.1 μg of ODN molecules (5′ tct ccc agc gtg cgc cat 3′) (Sigma-Proligo) in a final volume of 10 μl for 1 h at room temperature. After incubation, the samples were loaded into the wells of 1% agarose gel (stained with 0.5 μg/mL of ethidium bromide) in 1×TBE (Tris-borate-EDTA) buffer for electrophoresis. Same amount of ODN molecules alone were loaded for comparison (lane 1). The electrophoresis was run for 1 h at 80 mV. The DNA bands were visualized under a UV transilluminator and the gel was then fixed, dried and photographed under UV light.

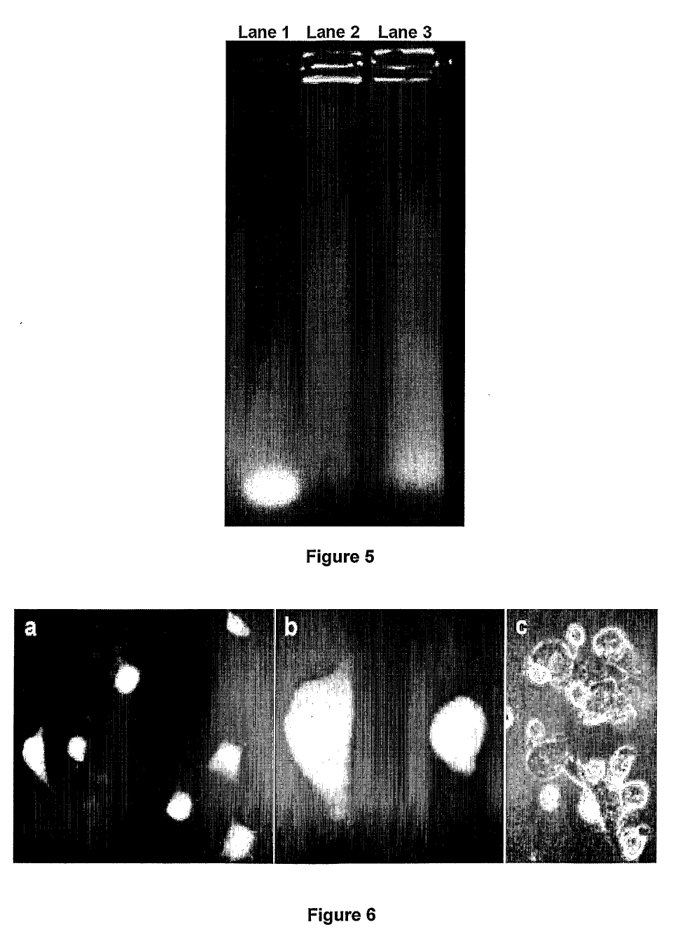

This procedure was based on the observation that the formation of ODN and protein/or lipid complexes usually reduces the electrophoretic mobility of the ODN component in the gel matrix. As expected from the fluorescence microscopic and TEM studies, the gel tests revealed that the amount of mobile ODNs (i.e. dissociated from the starved cubic mitochondria) detected by applying an electrical field was much less than that found in the preparation of mouse liver mitochondria (FIG. 5). The mitochondria with cubic membrane organization were able to retard ODNs mobilization (lane 2) towards the positive pole as compared to the same amount of pure ODNs (lane 1) and ODNs incubated with isolated mouse liver mitochondria (lane 3). This proves that mitochondria with membranes of cubic structure retain ODN molecules against electrical field dissociation. The results illustrate a representative experiment of at least three others with similar findings, showing one of duplicate samples.

### Example 4

### Transfection

In vitro transfection was used to test the uptake of fragmented ODN-cubic membrane complexes in targeted cells. MCF-7 cells (human breast adenocarcinoma) cell line was obtained from American Type Culture Collection (ATCC Cat. No: HTB-22). MCF-7 cells (1×10⁵) were seeded onto 6-well plates in Dulbecco's Modification of Eagle's Medium (DMEM), supplemented with 10% Fetal Bovine Serum (FBS) and 1% ampicillin. The seeded MCF-7 cells were left for 24 h prior to the transfection to reach a confluency of around 50% to 60% on the day of transfection. After 24 h, the medium was removed and replaced with fresh DMEM (1 ml).

Two dishes of 10 days starved amoeba were harvested and mitochondria were isolated using the procedures described in Example 1.50 μl of fluorescein-tagged ODNs according to Example 1 at a concentration of 1.2 μg/μl was incubated with 50 μl of cubic mitochondria suspension (26 μg/μl) for 1 h. EDTA (ethylenediaminetetraacetic acid) was added into the mixture to maintain a final concentration of 10 mM which was found to be crucial for the preservation of cubic structure. After incubation, the mixture was sonicated in a water bath (Soniclean Ultrasonic, Model 250HT 6Iw, Australia) at 37° C. for 6 min at the high amplitude setting. As cubic mitochondria are large (1-5 μm in diameter) particles (Deng and Mieczkowski, 1998), ultrasound sonication of starved mitochondria containing ODNs led to fragmentation, forming stable submicron-sized particles containing ODNs (“ODN delivering particle”). MCF-7 cells were washed with FBS-free DMEM 3 times and 1 ml of FBS-free DMEM was added into the well. The ODN delivering particle was then added into one well.

There were three negative controls. One with MCF-7 cells incubated with 0.2% BSA isolation medium, another with MCF-7 cells incubated with cubic mitochondria suspension and a third with MCF-7 cells incubated with only ODNs at the same amount as the transfection well. The cells were incubated in the incubator with 5% CO₂ at 37° C. for 5 h. Thereafter cells were washed with 1×PBS 3 times and the transfection medium was replaced with fresh DMEM. Some of the cells were collected and fixed for TEM studies (FIG. 3). The remaining cells were viewed 5 h post-transfection under the fluorescence microscope for the uptake of tagged-ODNs.

The results in FIGS. 6(a) and (b) show that in the cells incubated with ODN delivering particles, tagged-ODNs were found in both the cytoplasm and nucleus. This was further confirmed by the results obtained using the phase-contrast fluorescence microscope FIG. 6(c). The green fluorescence-tagged ODNs, observed in the cytoplasm and the nucleus (a and c: 64×, b: 200×) are shown as bright spots in contrast to the grey background in (a) to (c). The results illustrate a representative experiment of at least three to five others with similar findings.

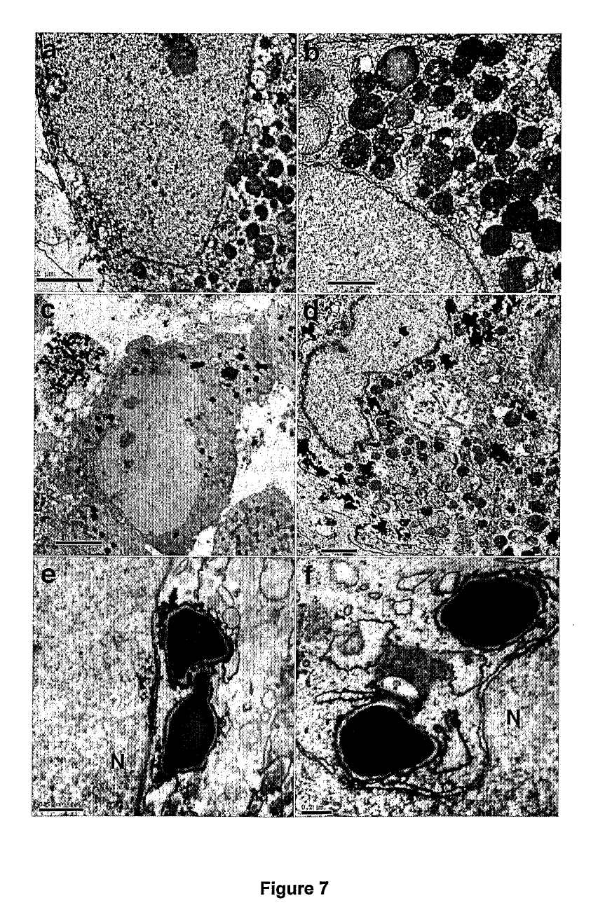

The ODN delivering particle internalization procedure was further examined under TEM at different time points, and data showed several cytoplasmic inclusions (FIG. 7(a)-(f)), including some adjacent to the target nucleus (FIG. 7(c)). There were no inclusion bodies observed in MCF-7 cells without the ODN treatment (FIG. 7(a)) or MCF-7 cells incubated with naked ODNs (FIG. 7(b)). However, multiple inclusion bodies were observed in those MCF-7 cells that were transfected with cubic membrane-mediated ODNs complexes for 5 h (c and d). Multiple ‘electron-dense’ inclusion bodies were observed in the cytoplasm and some targeted the nucleus (N). At higher magnification, membrane association was frequently observed between electron-dense inclusion bodies and nuclear membranes (e and f). Evidently, from the data obtained, it appears that the mitochondrial fragments offer robust delivery and release vehicles for at least short strands of DNA.

### Example 5

### Lipid Extraction from Chaos Cells

The lipid was extracted from fed and 7d-starved Chaos cells and analyzed for phospholipids and fatty acid profiles. For efficient extraction of phospholipids (including cardiolipin, CL) from Chaos cells, a few extraction approaches based on Folch (Folch et al., 1957) or Bligh and Dyer (Bligh and Dyer, 1959) methods were carried out and lipid profiles obtained were further analyzed. A two-step extraction protocol was applied to maximally extract the lipids from Chaos cells (cardiolipin was used as an indicator). The harvested Chaos cells according to Example 1 was homogenized in chloroform:methanol (1:2, v:v) mixed solvent. The mixture was vortexed for 30 sec and lipids extracted at 8° C. at 1200 rpm in a Thermomixer (Thermoshakder, TS-100, BIOSAN) for 10 min followed by the addition of 0.3 ml chloroform and 0.2 ml 1M KCl into each tube. The mixture was then vortexed for 30 min and left to incubate at 4° C. for 1 min. The mixture was then high-speed centrifuged at 9,000 rpm for 2 min to separate the phases. The lower phase was transferred to a clean tube (Extract 1). 0.6 ml of chloroform was then added to the remaining mixture in the tube for a second extraction, to obtain Extract 2. Extract 1 and 2 were then combined and dried using speedvac to obtain the dried lipids, which were kept in a freezer at −20° C. The dried lipids were re-dissolved in chloroform:methanol (1:1, v:v) prior to high-performance liquid chromatography (HPLC), Electrospray ionization (ESI) and Mass Spectrometry (MS) analysis. Nine batches of paired samples (fed vs. 7 days starved) were systematically investigated and analyzed using the above-mentioned lipid extraction protocol.

### Lipid Profile Analysis by HPLC/ESI/MS

Both fed and 7 days starved amoeba lipid extracts were directly infused for positive ESI/MS analysis. Phosphatidylcholine (PC) was proven to be ionized at both positive and negative modes, although it ionized better at the positive mode. However, the other major phospholipids such as phosphatidylethanolamine (PE), phosphatidylinositol (PI) and cardiolipin (CL) were not well ionized at the positive mode. Therefore, it was observed that most major phospholipids were found to be well characterized and measured at the negative mode. Negative ESI/MS was thus utilized for lipid profiling assay of the Chaos cells, and positive ESI/MS was only used for further structural confirmation.

Both negative ESI/MS/MS and HPLC/ESI/MS/MS analyses were carried out under the conditions similar to the above-mentioned protocol except for collision energies. Most of the ion peaks, especially the major peaks and those with significant changes after starvation were further investigated using tandem mass spectrometry either through ESI/MS/MS or HPLC/ESI/MS/MS with collision energy from 25-50 ev, and argon was used as the collision gas. HPLC/ESI/MS/MS was necessary for structural identification of many small ion peaks, e.g. m/z 853, 867, 866, 868, 804, etc., as HPLC separation would reduce the interference of other isobaric compounds or isotopic interferences of neighbour ion peaks. Direct ESI tandem mass spectrometry was also carried out at the positive mode for structural elucidation of individual ions, and the results were compared with corresponding ions obtained at the negative mode.

Data Processing for Comparative Study of Lipid Profiles of Amoeba Chaos Cells (Fed Vs. 7d-Starved Chaos Cells)

As numerous compounds were present in the amoeba lipid extract, it was not easy to use traditional HPLC technique for the lipid analysis (quantification or semi-quantification). In order to simplify data processing for the comparative study, the mass spectrometry during the elution period of most lipids were combined, i.e. from 3 min to 20 min in this study. The combined mass spectrometry included more than ten thousand sets of 2-dimensional data (m/z vs. intensity). Based on the sum of all ions detected, the intensities of both fed and starved samples were normalized for the comparison. In the final form of data presentation, only a few hundred peaks (most of isotopic peaks were not included), which could be judged as peaks in mass spectra, were further normalized to show the percentage contribution of individual ions to the sum of detectable species. This is provided in Table 1 below. As can be seen from Table 1, there are still many compounds/lipids that have not been identified (designated with “?”).

TABLE 1
A comparative study of detailed lipid profiles in amoeba Chaos
under fed and 7 days starved conditions using HPLC/ESI/MS.

| m/z | species | fed % | starved % | increase % |
|---|---|---|---|---|
| 436.4 | lysoPE(16:0p; 16:1e) | 1.02 | 2.19 | 114.06 |
| 450.4 | ? | 0.20 | 0.32 | 59.72 |
| 451.6 | ? | 0.26 | 0.32 | 23.62 |
| 457.3 | ? | 0.14 | 0.19 | 33.28 |
| 459.3 | ? | 0.18 | 0.31 | 68.30 |
| 462.5 | lysoPE(18:0e) | 0.20 | 0.45 | 118.56 |
| 464.4 | lysoPC(16:0p; 16:1e) | 0.68 | 1.82 | 169.35 |
| 465.4 | ? | 0.79 | 0.98 | 23.88 |
| 473.4 | ? | 1.05 | 1.13 | 7.94 |
| 482.5 | ? | 0.17 | 0.23 | 34.08 |
| 485.3 | ? | 0.16 | 0.25 | 49.99 |
| 492.5 | ? | 0.34 | 0.38 | 13.40 |
| 507.6 | ? | 0.34 | 0.38 | 12.95 |
| 510.4 | ? | 0.40 | 0.24 | −39.42 |
| 514.5 | ? | 0.15 | 0.41 | 169.48 |
| 516.5 | ? | 0.40 | 0.58 | 46.70 |
| 517.1 | ? | 0.65 | 0.38 | −41.71 |
| 517.5 | ? | 0.19 | 0.28 | 49.73 |
| 524.4 | ? | 8.00 | 4.76 | −40.52 |
| 529.6 | ? | 1.38 | 2.02 | 45.98 |
| 535.7 | ? | 0.31 | 0.41 | 33.24 |
| 542.5 | ? | 2.18 | 3.55 | 63.11 |
| 549.7 | ? | 0.27 | 0.30 | 11.93 |
| 554.5 | LysoPC(22:5) | 0.30 | 0.34 | 12.25 |
| 560.5 | ? | 5.60 | 5.53 | −1.16 |
| 562.4 | ? | 1.78 | 1.74 | −2.50 |
| 564.5 | ? | 0.43 | 0.58 | 35.15 |
| 572.6 | ? | 5.99 | 4.34 | −27.51 |
| 586.6 | ? | 1.37 | 0.96 | −30.16 |
| 590.6 | ? | 0.43 | 0.25 | −41.38 |
| 591.7 | ? | 0.38 | 0.37 | −2.74 |
| 600.6 | ? | 0.42 | 0.26 | −37.47 |
| 619.6 | hydroxylated lysoCL | 0.26 | 0.27 | 3.54 |
| | (temp assignment) | | | |
| 630.6 | hydroxylated lysoCL | 0.15 | 0.21 | 36.33 |
| | (temp assignment) | | | |
| 631.1 | hydroxylated lysoCL | 0.13 | 0.16 | 25.91 |
| | (temp assignment) | | | |
| 631.6 | hydroxylated lysoCL | 0.20 | 0.29 | 40.73 |
| | (temp assignment) | | | |
| 632.1 | hydroxylated lysoCL | 0.18 | 0.26 | 46.16 |
| | (temp assignment) | | | |
| 632.6 | hydroxylated lysoCL | 0.41 | 0.71 | 73.86 |
| | (temp assignment) | | | |
| 633.8 | hydroxylated lysoCL | 0.32 | 0.39 | 22.15 |
| | (temp assignment) | | | |
| 643.6 | hydroxylated lysoCL | 0.14 | 0.16 | 13.25 |
| | (temp assignment) | | | |
| 645.5 | LysoPI(22:5) | 0.17 | 0.28 | 62.76 |
| 674.7 | PC[30:0P(16:0/14:0, 15:/15:0)] | 0.49 | 0.91 | 85.79 |
| 682.7 | ? | 0.33 | 0.16 | −52.42 |
| 688.7 | PE[32:1(16:1/16:0)], | 0.17 | 0.28 | 64.81 |
| | PC[30:1A(16:1/14:0, 15:0/15:1)] | | | |
| 702.8 | PC[32:0P(16:0/16:0), | 0.29 | 0.40 | 38.46 |
| | 32:1E(18:0p/14:0)] | | | |
| 705.7 | Need further characterisation | 1.06 | 1.00 | −5.72 |
| 719.7 | PG[32:1)16:0/16:1], | 5.80 | 4.07 | −29.82 |
| | PA[38:6(22:5/16:1)] | | | |
| 724.7 | PC(30:0P(16:0p/14:0, 15:0p/15:0)], PE | 0.24 | 0.27 | 13.26 |
| 726.8 | PC[34:2P/34:3E(18:2/14:0, | 0.50 | 0.19 | −61.97 |
| | 18:3/14:0)], PE[36:2P, | | | |
| | 36:3e(18:1/18:1, 18:1/18:2)], | | | |
| | PC[30:0e(14:0/16:0)] | | | |
| 732.7 | PE[37:6p(22:6/15:0, 22:5/15:1)], | 0.16 | 0.33 | 99.99 |
| | PC[33:0A(18:0/15:0)] | | | |
| 734.7 | PE[37:5p(22:5/15:0] | 1.13 | 1.16 | 2.94 |
| 736.7 | PE(36:5A), TEMP | 0.27 | 0.23 | −14.05 |
| 746.7 | PE[38:6P(22:6/16:0)], | 0.78 | 0.76 | −2.70 |
| | PE[36:0(18:0/18:0)], | | | |
| | PC[34:0(18:0/16:0)] | | | |
| 748.8 | PE[38:5P(16:0p/22; 5)] | 13.05 | 10.46 | −19.83 |
| 762.8 | PC[36:6a(22:5/14:1, 18:3/18:3, | 0.31 | 0.43 | 36.10 |
| | 22:6/14:0); PE[38:6A(20:4/18:2, | | | |
| | 20:3/18:3, 22:5/16:1, 22:6/16:0)] | | | |
| 764.8 | PC[36:5a(22:5/14:0, | 0.47 | 0.65 | 39.59 |
| | 18:3/18:2); PE[38:5A(22:5/16:0, | | | |
| | 20:4/18:1, 20:5/18:0)] | | | |
| 766.8 | PC[36:4a(20:4/16:0), | 0.29 | 0.23 | −20.08 |
| | PE[38:4a(20:4/18:0, 20:3/18:1)] | | | |
| 773.7 | CL(80:14) | 0.33 | 0.29 | −11.14 |
| 774.2 | CL(80:14) | 0.33 | 0.29 | −11.14 |
| 774.8 | CL(80:13), PC[38:6p(22:5/16:1e)] | 0.87 | 1.16 | 32.38 |
| 775.3 | CL(80:13) | 0.38 | 0.37 | −2.53 |
| 775.7 | CL(80:12) + PC isomer | 1.57 | 1.66 | 5.41 |
| 776.2 | CL(80:12) | 0.94 | 0.72 | −23.73 |
| 776.8 | PC[38:5P/38:6E(22:5/16:0p, | 1.50 | 3.32 | 121.89 |
| | 22:5/16:1e)] | | | |
| 777.3 | CL(80:11) + PC isotope | 0.16 | 0.64 | 292.45 |
| 778.8 | PC(38:4P; 38:5E) + CL | 0.56 | 0.67 | 19.19 |
| 785.7 | CL(82:16) | 0.16 | 0.19 | 13.94 |
| 786.2 | CL(82:16) | 0.12 | 0.08 | −34.34 |
| 786.7 | CL(82:15) | 0.56 | 0.40 | −27.87 |
| 787.8 | CL(82:14) | 0.74 | 0.60 | −18.86 |
| 788.8 | CL(82:13) | 1.06 | 0.42 | −60.10 |
| 789.8 | CL(82:12) | 0.26 | 0.23 | −13.26 |
| 790.8 | PC[38:6a(22:5/16:1)], | 1.01 | 1.34 | 32.60 |
| | PE[40:6a(22:5/18:1)] | | | |
| 792.8 | PC[38:5a(22:5/16:0, 20:4/18:1)], | 0.72 | 0.85 | 17.18 |
| | PE[40:5a(22:5/18:0)] | | | |
| 794.8 | PC[38:4a(20:3/18:1, 20:4/18:0, | 0.38 | 0.49 | 27.33 |
| | 20:2/18:2)] | | | |
| 798.8 | CL(84:17) | 0.48 | 0.27 | −44.81 |
| 799.8 | CL(84:16) | 0.32 | 0.32 | 0.08 |
| 800.3 | CL(84:16) | 0.29 | 0.32 | 12.93 |
| 800.8 | PC[40:6p] | 0.28 | 0.30 | 7.58 |
| 802.8 | PC[40:6p(22:5/18:1p, 22:6/18:0p), | 0.20 | 0.22 | 11.27 |
| | 40:7e(22:5/18:2e, 22:6/18:1e, | | | |
| | 20:4/20:3)] | | | |
| 804.8 | PC[40:6e(22:5/18:1e, 20:3/20:3, | 0.36 | 0.53 | 46.85 |
| | 20:4/20:2, 22:6/18:0), | | | |
| | 40:5p(22:5/18:0)] | | | |
| 806.9 | PC[40:5e(23:5/17:0e, 22:5/18:0) | 0.20 | 0.35 | 71.12 |
| | 20:3/20:3, 20:4/20:2, 22:6/18:0), | | | |
| | PC[37:5a(22:5/15:0, 23:5/14:0)], | | | |
| | PE[39:5A(22:5/17:0, 23:5/16:0)] | | | |
| 808.8 | PC derivatives | 0.14 | 0.24 | 74.22 |
| 810.8 | PC derivatives TEMP | 0.11 | 0.18 | 65.94 |
| 812.8 | PC[36:6a(22:5/14:1, 18:3/18:3, | 0.31 | 0.49 | 54.98 |
| | 22:6/14:1)], PE[42:9a(22:5/20:4)] | | | |
| 814.8 | PC[40:8a (22:5/18:3, 20:4/20:4, | 0.34 | 0.33 | −3.40 |
| | 22:6/18:2)], PC[36:5a (22:5/14:0)], | | | |
| | PE[42:8a(22:5/20:3)] | | | |
| 816.8 | PC[40:7a (22:5/18:2, 20:4/20:3, | 0.27 | 0.23 | −16.95 |
| | 22:6/18:1)], PC[36:4a (20:4/16:0, | | | |
| | 18:1/18:3, 18; 2/18:2, 22:4/14:0)] | | | |
| 818.8 | PC[40:6a (22:5/18:1, 20:4/20:2, | 0.44 | 0.68 | 54.28 |
| | 20:3/20:3)], PC[36:3a(20:3/16:0)] | | | |
| 820.8 | PC[40:5A(22:5/18:0)], | 0.21 | 0.27 | 33.89 |
| | PC[36:2a(18:1/18:1)], | | | |
| | PE[38:5a(22:5/16:0)] | | | |
| 824.8 | PC[38:6P(16:0p/22:6)] | 0.19 | 0.32 | 63.41 |
| 826.8 | PC[38:5P(16:0p/22:5)] | 0.37 | 0.81 | 118.92 |
| 828.8 | oxidized PC[38:5p(22:5/16:0p.OCL)] | 0.24 | 0.41 | 69.58 |
| 836.8 | PC derivatives | 0.24 | 0.30 | 24.35 |
| 838.8 | PC[42:10a (22:5/20:5, 22:6/20:4)]; | 0.22 | 0.33 | 46.40 |
| | PC[38:7a(22:5/16:2, 20:4/18:3, | | | |
| | 20:5/18:2)] | | | |
| 840.8 | PC[42:9a]; PC[38:6a] | 0.24 | 0.24 | 2.37 |
| 842.8 | PC[42:8a(22:5/20:3)]; | 0.26 | 0.28 | 5.38 |
| | PC[38:5a(22:5/16:0, 20:4/18:1)] | | | |
| 844.8 | PC[42:7a(22:5/20:2)]; | 0.15 | 0.19 | 30.01 |
| | PC[38:4a(20:3/18:1, 20:2/18:2, | | | |
| | 20:4/18:0)] | | | |
| 848.9 | PC related | 0.13 | 0.26 | 101.40 |
| 849.9 | PI temp | 0.29 | 0.22 | −25.57 |
| 850.9 | PC related | 0.26 | 0.78 | 199.02 |
| 852.9 | PC related | 0.19 | 0.23 | 23.21 |
| 853.8 | PI[36:6a(22:5/14:1)] | 0.13 | 0.20 | 56.57 |
| 854.8 | PC[40:5p(18:0p/22:5)] | 0.20 | 0.25 | 24.36 |
| 864.8 | PC[44:11a(22:5/22:6)], PC[40:8a | 0.16 | 0.20 | 22.62 |
| | (22:5/18:3, 20:4/20:4, 22:6/18:2)] | | | |
| 865.8 | PI[37:7a[22; 5/15:2)] | 0.13 | 0.18 | 32.65 |
| 866.8 | PC[44:10a(22:5/22:5)], PC[40:7a | 0.43 | 0.81 | 89.24 |
| | (22:5/18:2, 22:6:18:1) | | | |
| 867.8 | PI[37:6a[22; 5/15:1)] | 0.74 | 1.80 | 144.35 |
| 868.8 | PC[44:9a(22:5/22:4)]; PC[40:6a | 0.51 | 0.97 | 91.55 |
| | (22:5/18:1)] | | | |
| 869.8 | PI[37:5a[22; 5/15:0)] | 0.67 | 0.58 | −13.81 |
| 870.8 | PI isotope + PC related | 0.42 | 0.34 | −19.47 |
| 878.9 | PC [45:11a(23:6/22:5)] | 0.16 | 0.20 | 25.36 |
| 886.8 | PC related | 0.09 | 0.12 | 34.99 |
| 888.9 | PC[42:10a (22:5/20:5, 22:6/20:4)] | 0.14 | 0.13 | −11.16 |
| 890.9 | PC[42:9a(22:5/20:4)] | 0.18 | 0.22 | 24.41 |
| 892.9 | PC[42:8a(22:5/20:3, 20:4/22:4)] | 0.17 | 0.25 | 43.26 |
| 914.8 | PC[44:11a(22:5/22:6)] | 0.13 | 0.16 | 26.41 |
| 916.9 | PC[44:10a(22:5/22:5)] | 1.06 | 1.35 | 27.25 |
| 918.8 | PC[44:9a(22:5/22:4)] | 0.18 | 0.29 | 62.33 |
| 940.9 | PC related | 0.07 | 0.16 | 134.17 |
| 955.8 | PI[44:11(22:5/22:6)] | 0.31 | 0.24 | −23.09 |
| 957.8 | PI[44:10(22:5/22.5)] | 11.61 | 7.56 | −34.84 |
| 1210.0 | MoloLysoCL(57:6) | 0.16 | 0.20 | 20.71 |
| 1222.1 | MoloLysoCL(58:7) | 0.28 | 0.26 | −7.45 |
| 1246.1 | MoloLysoCL(60:9) | 0.05 | 0.16 | 197.55 |
| 1270.2 | MoloLysoCL(62:11) | 0.14 | 0.05 | −64.43 |

Assignment of phospholipids according to tandem mass spectra of individual ions.

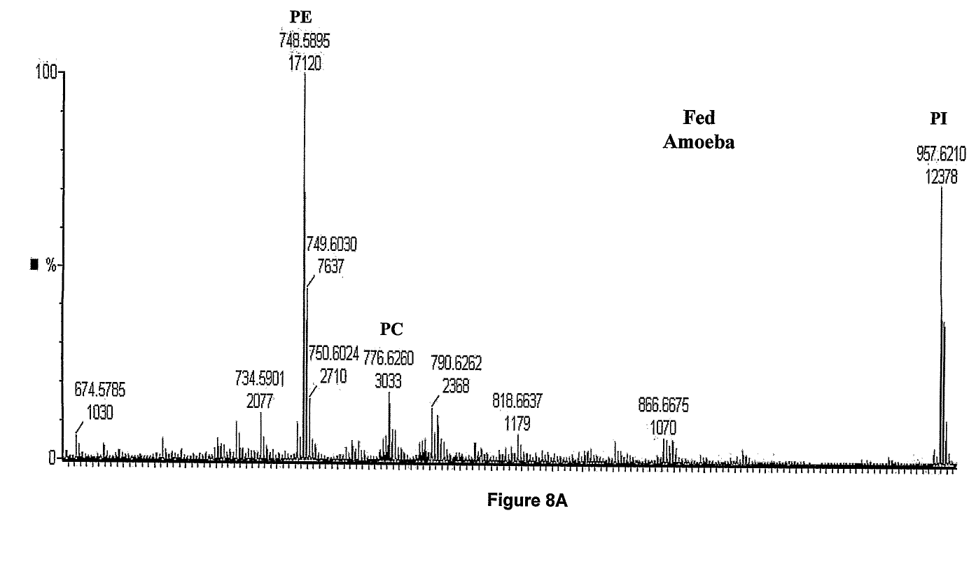

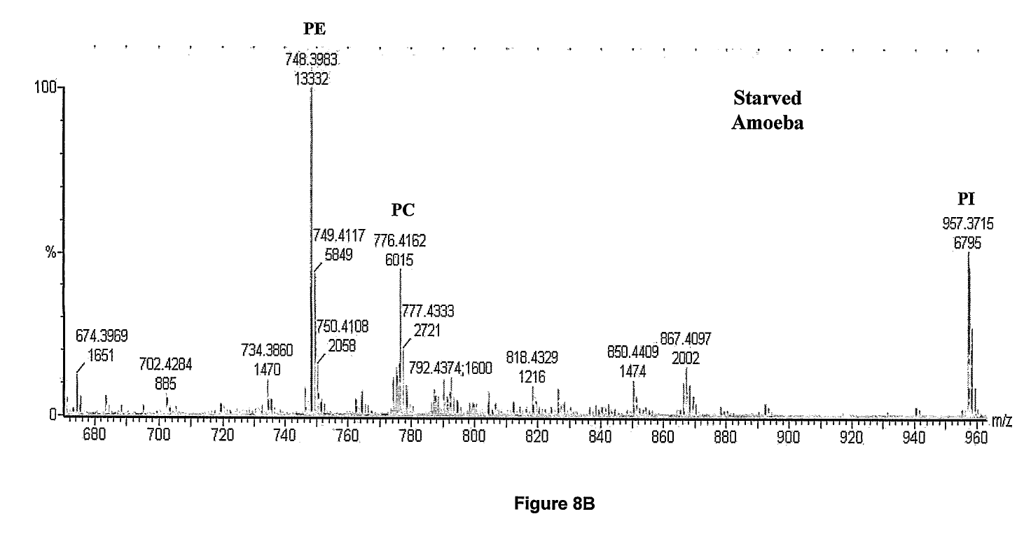

The graphical representation within the range 600-1000 m/z of Table 1 is shown in FIGS. 8A and B which provides the results from the mass spectrometry of all the lipids of the Chaos cell. The overall lipid profiles of both fed (A) and 7 days starved (B) Chaos samples were the same except for the alterations in the amounts of the lipids. Three major species PE, PC and PI (748, 776 and 957 m/z respectively) in the range 600-1000 m/z are highlighted in the lipid profiles of both fed and starved samples.

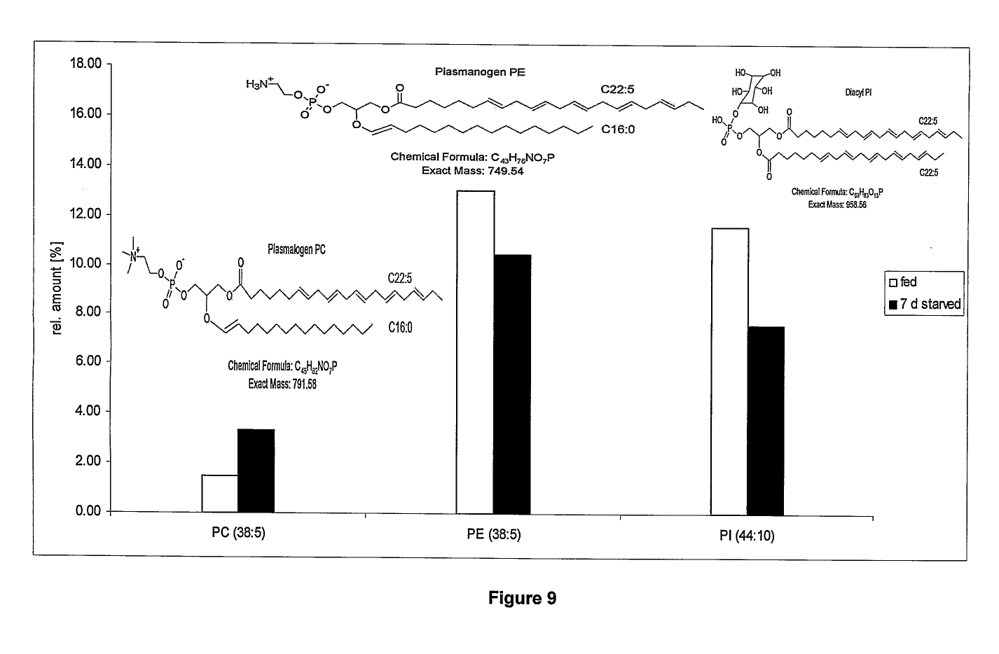

A further characterization of the three major phospholipids—PC (38:5), PE (38:5) and PI (40:10) of both fed vs. 7 days starved amoeba Chaos samples are shown in FIG. 9. The corresponding chemical structure of each lipid class was determined and displayed above each lipid class. As can be seen in FIG. 9, there was a significant increase in plasmalogen PC (22:5/16:0), and a decrease in both plasmalogen PE (22:5/16:0) and diacyl-PI (22:5/22:5) upon Chaos starvation. These three compounds may thus play an essential role in the formation of membranes with cubic structure.

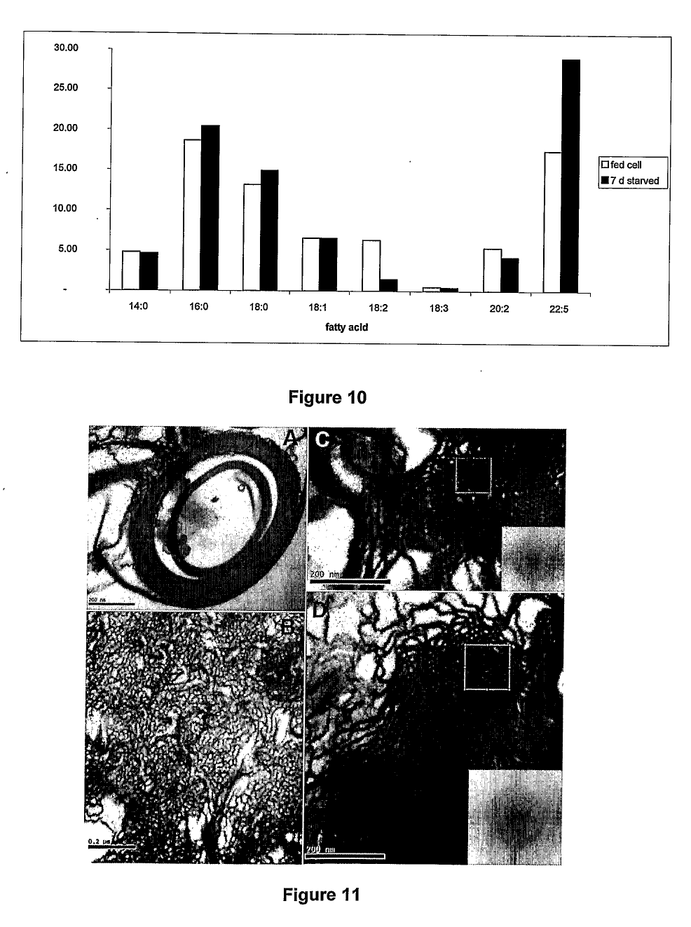

The relative distribution of fatty acids of fed and 7 days starved Chaos cells was also studied and the results are shown in FIG. 10. As can be seen in FIG. 10, linoleic acid (C18:2) significantly decreased in the 7 days starved Chaos cells compared to the fed Chaos cells. There was also a significant increase in docosapentaenoic acid (C22:5) in total amoeba lipid extracts, upon 7 days of starvation compared to the C22:5 fatty acids of the fed Chaos cells. It is clearly demonstrated that Chaos cells responded to starvation by having more highly polyunsaturated fatty acids in their membrane lipids. The highly polyunsaturated fatty acid content in starved amoeba may partly explain the ability for mitochondrial cubic membranes transformation under starvation stress condition.

### Example 6

### Liposomal Construction

Liposomal construction was used to investigate the possibility of the lipids self-assembling into lipid membranes of cubic structure without interference from protein. The extracted lipids prepared according to Example 5 from fed and 7 days starved whole Chaos cells were used for liposomal preparation followed by a TEM ultrastructural study. The dried lipid film from Example 5 was resuspended in 1 ml of miniQ water. The lipids and water were mixed and sonicated well to prepare liposomes which were then freezed in liquid nitrogen and thawed in the water bath at room temperature. The procedure was repeated three times. The liposomal pellets were then obtained by centrifugation at 15,000 rpm and fixed in 2.5% glutaraldehyde for further TEM study.

### TEM Ultrastructural Study and Image Analysis

Two sets of liposomal pellets (from fed vs. 7 days starved Chaos cells) were studied by the conventional TEM technique. The 2.5% glutaraldehyde fixed liposomal pellets were fixed a second time with 1% osmium tetroxide followed by a series of ethanol dehydration, and finally embedded in Epon-Araldite. The ultra-thin sections (70-90 nm) were cut and viewed under a Joel 1010 TEM. The patterned membrane domains in TEM micrographs were further examined and analyzed using fast Fourier transformation (FFTs) implemented on an ImageJ (NIH, USA) image analysis system.

The results of TEM study are shown in FIG. 11. The liposomes prepared from fed amoeba lipids extract tended to form multi-lamellar (A) or sponge membrane structures (B); while the liposomes made from lipids extracted from 7 days starved Chaos cells displayed cubic (C) or hexagonal (D) organization. Fast Fourier transformation (FFT) analysis confirmed symmetrical organization (displayed in lower right of panel of C and D). The results showed that lipids play a major role in biological membrane phase transition. This is contradictory to the widely-accepted idea, which is that membrane-bound proteins are the major players of membrane transformation.

### Example 7

### Isolation of Chaos Mitochondria and Mitochondrial Protein Profiling

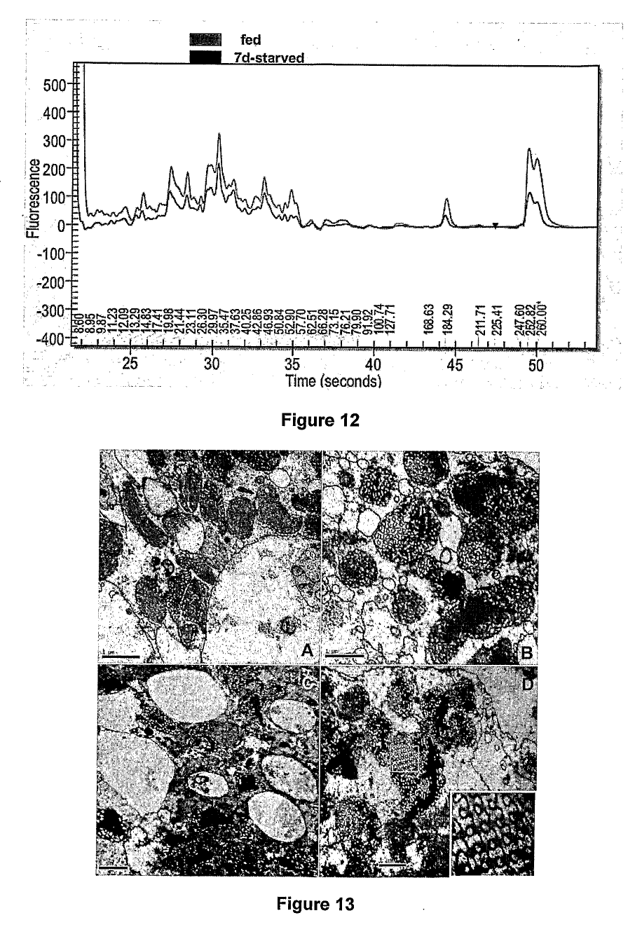

Samples of fed and 7 days starved amoeba mitochondria were isolated using the methods described in Example 1. The isolated amoeba mitochondrial samples (fed vs. 7 days starved) containing proteins (1 μg/1 μl) were used for protein profile analysis by Automated Gel Electrophoresis System Experion™ (BioRad) using Protein 260 Plus LabChip® kit. The mitochondrial protein profiles of amoeba Chaos cells (fed vs. 7 days starved) are shown in FIG. 12. The results of the protein profiling confirm the results of Example 6, which reveal that membrane-bound proteins are not the major players of membrane transformation.

### Example 8

### The Effects of Continuous Addition of Docosapentaenoic Acid (DPA) (C22:5) into Amoeba Culture Medium: TEM Study

All-cis-4,7,10,13,16 Docosapentaneoic Acid (DPA, C22:5n-6) (Sigma) and Cis-7,10,13,16,19 DPA (C22:5n-3) (Sigma) were dissolved in Diethyl Ether and added to the amoeba culture media at a final concentration of 0.1 mM. DPA (n-3) and DPA (n-6) were added separately after amoeba washing and in the presence of the food organisms (Paramecium multimicronucleatum). The procedure was performed every alternative day for a total of 3 times according to the regular feeding schedule. At day 5 post-DPA treatment Chaos cells were fixed in 2.5% glutaraldehyde for TEM processing according to the procedure described in Example 6.

The TEM results on the effects of continuous feeding of docosapentaenoic acids (DPA, 22:5) to amoeba Chaos with the presence of their food organisms Paramecium are shown in FIG. 12(A) Fed control, (B) 7 days starved control (C) Chaos cells with continuous feeding DPA (n-3) for 5 days; and continuous feeding DPA (n-6) (D) to amoeba Chaos for 5 days. A continuous feeding of DPA (n-6) to fed Chaos cells enhanced their inner mitochondrial membrane transformation from random tubular (A) to cubic organization (D); while feeding DPA (n-3) did not have the same drastic effects on the inner mitochondrial membrane organization. These observations strongly suggest the key role of DPA (n-6) but not (n-3) in the cubic membrane formation in the mitochondria of amoeba Chaos.

### REFERENCES

Tan et al., (2005) Protistology. 4(2): 185-190
Deng and Mieczkowski, (1998) Protoplasma, 203: 16-25.
Bradford, (1976). Anal Biochem. 72:248-54.
Almsherqi et al., (2006). J Cell Biol 173:839-844.
Folch et al., (1957) J Biol. Chem. 226: 497
Bligh and Dyer, (1959). Can. J. Biochem. Physiol. 37, 922

1.-42. (canceled)

43. An agent delivery vehicle comprising at least one lipid membrane of cubic structure.

44. The agent delivery vehicle according to claim 43, wherein the lipid membrane comprises at least one lipid selected from the group consisting of fatty acid C22:5, plasmalogen phosphatidylcholine (PC), plasmalogen phosphatidylethanolamine (PE) and diacyl phosphatidylinositol (PI).

45. The agent delivery vehicle according to claim 43, further comprising at least one foreign agent.

46. The agent delivery vehicle according to claim 45, wherein the agent delivery vehicle is for use in delivery of the foreign agent into at least one cell in vivo or in vitro.

47. The agent delivery vehicle according to claim 43, wherein the foreign agent is selected from the group consisting of hydrophilic drug, hydrophobic drug, nucleic acid, polypeptide, polysaccharide, virus, and vitamin.

48. The agent delivery vehicle according to claim 43, wherein the lipid membrane is made from at least one lipid of at least one organelle comprising at least one membrane of cubic structure.

49. The agent delivery vehicle according to claim 48, wherein the organelle is selected from the group consisting of chloroplast, Golgi apparatus, mitochondrion, nucleus, rough endoplasmic reticulum (RER), smooth endoplasmic reticulum (SER), vacuole, and vesicle.

50. The agent delivery vehicle according to claim 48, wherein the organelle is isolated from at least one amoeba.

51. The isolated organelle according to claim 50, wherein the amoeba is Chaos carolinense.

52. The agent delivery vehicle according to claim 43, wherein the agent delivery vehicle is in the form of liposome.

53. A method of preparing one agent delivery vehicle according to claim 43 comprising contacting at least one lipid selected from the group consisting of fatty acid C22:5, plasmalogen phosphatidylcholine (PC), plasmalogen phosphatidylethanolamine (PE) and diacyl phosphatidylinositol (PI), with at least one aqueous medium.

54. A method of delivery of at least one foreign agent comprising contacting an agent delivery vehicle comprising at least one lipid membrane of cubic structure with at least one cell in vivo or in vitro.

55. The method according claim 54, wherein the method is for treating at least one subject comprising administering to the subject at least one agent delivery vehicle comprising at least one lipid membrane of cubic structure and at least one foreign agent.

56. The method according to claim 55, wherein the treatment is gene therapy treatment.

57. The method according to claim 55, wherein the administering is through inhalation, oral injection, surgical injection, rectal absorption, intravenous, subcutaneous or intramuscular means.
